(19) United States
(12) Patent Application Publication
(10) Pub. No.: US 2018/0211723 A1
(43) Pub. Date: Jul. 26, 2018

(54) PERSONALIZED NUTRITIONAL AND METABOLIC MODIFICATION SYSTEM

(71) Applicants: Katie Coles, Tampa, FL (US); Mark Springer, Tampa, FL (US)

(72) Inventors: Katie Coles, Tampa, FL (US); Mark Springer, Tampa, FL (US)

(21) Appl. No.: 15/934,549

(22) Filed: Mar. 23, 2018

Related U.S. Application Data
(63) Continuation-in-part of application No. 15/183,072, filed on Jun. 15, 2016.
(60) Provisional application No. 62/175,605, filed on Jun. 15, 2015.
(60) Provisional application No. 62/475,652, filed on Mar. 23, 2017.

(51) Int. Cl. G16H 20/60; G16H 10/60

(52) U.S. Cl. CPC G16H 20/60; G16H 40/63; G16H 10/60

(57) ABSTRACT

A personalized nutritional and metabolic modification system is described. The personalized nutritional and metabolic modification system includes a processor and a visual interface, which through a software program and in response to a user data set provides an avatar which transforms its appearance in response to changes in the user data set over time. A food selector component of the software program provides customized meal plans according to the characteristics and goals of the particular individual using the system by using evidence-based formulae to develop and adjusts a comprehensive nutritional plan in response to the individual's goals and changing activity. Changes in appearance of the avatar in response to changes in the user data over time provide feedback for tracking the user's progress through the visual interface.

### CROSS REFERENCE TO RELATED APPLICATION'S

This application is a continuation-in-part of the earlier U.S. Utility Patent Application entitled “PERSONALIZED NUTRITIONAL AND METABOLIC MODIFICATION SYSTEM,” Ser. No. 15/183,072, filed Jun. 15, 2016, which claims priority to U.S. Provisional Patent Application entitled “PERSONALIZED NUTRITIONAL AND METABOLIC MODIFICATION SYSTEM,” Ser. No. 62/175,605, filed Jun. 15, 2015, and this application claims priority to U.S. Provisional Patent Application entitled “PERSONALIZED NUTRITIONAL AND METABOLIC MODIFICATION SYSTEM,” Ser. No. 62/475,652, filed Mar. 23, 2017, the disclosures of which are hereby incorporated entirely herein by reference.

### BACKGROUND OF THE INVENTION

### Technical Field

This invention relates to a nutritional system, and more particularly to a personalized nutritional and metabolic modification system.

### State of the Art

Persons wishing to embark on a weight loss program associated with fitness goals, such as increasing muscle tone, increasing muscle mass, and losing body fat, often spend many hours researching the various weight-loss and fitness plans available on the Internet, and elsewhere in the public domain. Information from these sources is often conflicting and may or may not be based on scientific study. Additionally, some persons spend substantial amounts of money on fitness classes, personal nutritionists, meal planning and preparation, personal trainers, and dietary supplements. The particular regimen of diet/nutrition, dietary supplements, and exercise regimens is chosen from a staggering number of possible choices and combinations. Thus, making these choices time consuming, often expensive, and prone to error when based upon non-scientific or other misinformation. These choices are often made without the person having knowledge that the choice has a scientific basis for its purported utility. Also, changing requirements of a nutritional and exercise program as an individual's metabolism changes in response to weight loss, changes in body composition, and increasing fitness may not be a consideration in the chosen program.

There is a need for an efficient and comprehensive nutritional system that is personalized, evidence-based, automated, can be modified as the user progresses toward his or her particular goals, and which automatically adjusts to a user's changing metabolism and nutritional requirements. Additionally, an ongoing, long-term individualized maintenance program employed to harmonize with initial use of a compressive personalized nutritional system is desirable.

Accordingly, an invention is needed to provide a personalized nutritional and metabolic modification system that addresses these needs and overcomes the aforementioned issues.

### SUMMARY OF EMBODIMENTS

The invention relates to a personalized nutritional and metabolic modification system customized to the characteristics and goals of each specific individual using the system by using evidence-based methods to develop and adjust a comprehensive nutritional plan in response to the individual's goals and changing activity, and to display an avatar through a visual interface for tracking the individual's progress. Disclosed is a personalized metabolic and nutritional modification system comprising a user data set; a processor; a visual interface; a software program; an avatar, wherein the avatar transforms in appearance in response to changes in the user data set; and a food selector; wherein the food selector provides a food selection in response to changes in the user data set.

In some embodiments, the system further comprises a meal, wherein the meal comprises a macronutrient composition. In some embodiments, the system further comprising a preparation means, wherein the meal is prepared for the user. In some embodiments, the system further comprises a delivery means, wherein the prepared meal is delivered to the user.

Disclosed is a personalized macronutrient composition comprising a total caloric value; a carbohydrate weight; a protein weight; and a fat weight, wherein a food selector determines the total caloric value, the carbohydrate weight, the protein weight, and the fat weight in response to a user input and selects a food which comprises the total caloric value, the carbohydrate weight, the protein weight, and the fat weight.

An embodiment includes a personalized nutritional and metabolic modification system comprising: a user computing device for accessing the system, the user computing device comprising a visual interface, wherein the visual interface displays a graphical user interface for user interaction with the system including data input; and a computer server at a service provider, the computer server containing food recipe data, which computer server is coupled to the user computing device and programmed to: receive from the user computing device a signal indicating input of a user data set; automatically store the user data set and automatically determine a standardized ratio between carbohydrate weight, protein weight and fat weight in response to processing the stored user data set; automatically generate a recipe from stored food recipe data and transmit to the user computing device for display on the visual interface the recipe.

Another embodiment includes a personalized nutritional and metabolic modification system comprising: a user computing device for accessing the system, the user computing device comprising a visual interface, wherein the visual interface displays a graphical user interface for user interaction with the system including data input; and a computer server at a service provider, the computer server containing food recipe data, which computer server is coupled to the user computing device and programmed to: receive from the user computing device a signal indicating input of a user data set; automatically store the user data set; automatically generate an avatar having visually perceptible elements that are representative of the user's physical characteristics in response to processing the user data set and transmit to the user computing device for display on the visual interface the avatar; automatically determine a standardized ratio between carbohydrate weight, protein weight and fat weight in response to processing the stored user data set; automatically generate a recipe from stored food recipe data and transmit to the user computing device for display on the visual interface the recipe.

In some embodiments, the personalized macronutrient composition further comprises a standardized ratio between the carbohydrate weight, the protein weight, and the fat weight.

Another embodiment includes a personalized nutritional and metabolic modification system comprising: a user computing device for accessing the system, the user computing device comprising a visual interface, wherein the visual interface displays a graphical user interface for user interaction with the system including data input; and a computer server at a service provider, the computer server containing a machine learning master link, which computer server is coupled to the user computing device and the machine learning master link is programmed to: receive and automatically store current user information, wherein the current user information includes one of meals consumed by the user; timing of meals consumed; exercise performed, including type of exercise, length of exercise, heart rate, and/or calories burned; outcomes for the exercises performed; macronutrient outputs; amount of sleep; interruptions in sleep; stress incurred; or combinations thereof; process the user information and create current values associated with the current user information; automatically compare the current values generated regarding the current user information with historical values created from historical user information to determine the effect of the current user information with regard to the historical user information; and create and provide a user recommendation in response to the comparison of the current values and the historical values.

Another embodiment includes a personalized nutritional and metabolic modification system comprising: a user computing device for accessing the system, the user computing device comprising a visual interface, wherein the visual interface displays a graphical user interface for user interaction with the system including data input; and a computer server at a service provider, the computer server containing exercise recommendation module, which computer server is coupled to the user computing device and the exercise recommendation module is programmed to: receive and store exercises performed by trained professionals to establish reference points; build an automated periodization model for a variety of training protocols; receive and store a user's exercise data; process the data is fed into the system to gauge the user's overall performance and make adjustments and progressions; and send to the user computing device an exercise selection interface, wherein the exercise selection interface may include exercise programs and accessory work that can be selected from a menu, wherein the exercise programs are based on overall fitness goals and objectives of the user comprising at least one of “build muscle”, “get stronger”, “athletic performance”, “become more agile”, “get faster”, “run farther”, and combinations thereof.

Yet another embodiment includes a personalized nutritional and metabolic modification system comprising: a user computing device for accessing the system, the user computing device comprising a visual interface, wherein the visual interface displays a graphical user interface for user interaction with the system including data input; and a computer server at a service provider, the computer server containing a machine learning master link and an exercise recommendation module, which computer server is coupled to the user computing device and wherein: the machine learning master link is programmed to: receive and automatically store current user information, wherein the current user information includes one of meals consumed by the user; timing of meals consumed; exercise performed, including type of exercise, length of exercise, heart rate, and/or calories burned; outcomes for the exercises performed; macronutrient outputs; amount of sleep; interruptions in sleep; stress incurred; or combinations thereof; process the user information and create current values associated with the current user information; automatically compare the current values generated regarding the current user information with historical values created from historical user information to determine the effect of the current user information with regard to the historical user information; and create and provide user recommendations in response to the comparison of the current values and the historical values; and the exercise recommendation module is programmed to: receive and store exercises performed by trained professionals to establish reference points; build an automated periodization model for a variety of training protocols; receive and store a user's exercise data; process the data is fed into the system to gauge the user's overall performance and make adjustments and progressions; and send to the user computing device an exercise selection interface, wherein the exercise selection interface may include exercise programs and accessory work that can be selected from a menu, wherein the exercise programs are based on overall fitness goals and objectives of the user comprising at least one of “build muscle”, “get stronger”, “athletic performance”, “become more agile”, “get faster”, “run farther”, and combinations thereof.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a schematic diagram demonstrating elements of a personalized metabolic and nutritional modification system, in accordance with embodiments of the present invention;

FIG. 2 is a schematic diagramming the relationships between user activity levels of a personalized nutritional system, in accordance with embodiments of the present invention;

FIG. 3 is a schematic representation of a macronutrient composition for a personalized nutritional system, in accordance with embodiments of the present invention; and

FIG. 4 is a schematic representation of a user avatar for a personalized nutritional system, in accordance with embodiments of the present invention.

### DETAILED DESCRIPTION OF EMBODIMENTS OF THE INVENTION

Embodiments of this invention include a personalized metabolic and nutritional modification system. Personalized metabolic and nutritional modification system incorporates health and fitness goals of a user. Personalized metabolic and nutritional modification system includes a processor and a visual interface, which through a software program and in response to a user data set provides an avatar which transforms its appearance in response to changes in the user data set over time. A food selector algorithm of the software program provides customized meal plans according to the characteristics and goals of the particular individual using the system by using evidence-based formulae to develop and adjust a comprehensive nutritional plan in response to the individual's goals and changing activity. In some embodiments the user inputs food preferences and/or a range of likes and dislikes where the food selector algorithm can avoid dislikes and provide the customized meal plan as much as possible from the user's more likeable foods. Changes in appearance of the avatar in response to changes in the user data over time provide feedback for tracking the user's progress through the visual interface.

Details and descriptions of various embodiments of the invention are illustrated by the drawing figures and discussed in detail with reference to these figures herein below.

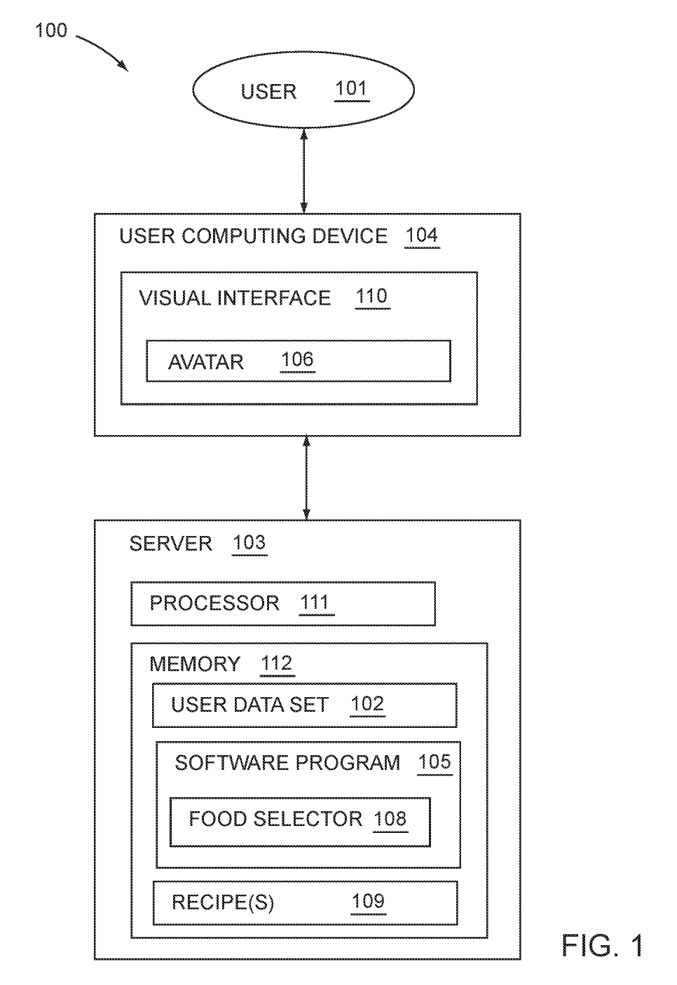

FIG. 1 is a schematic diagram demonstrating elements of a personalized metabolic and nutritional modification system 100. Metabolic and nutritional modification system 100 includes, in some embodiments, a user data set 102, a server 103 having a processor 111 and a memory 112, server 103 operating a software program 105 stored in memory 112 and executed by processor 111, the software program having a food selector 108 algorithm, and a user computing device 104. The server 103 and the user computing device 104 are coupled together, such as coupled through a network connection, including an Internet connection, a LAN, a WAN, a wired, a wireless and any type of network connection wherein data can be communicated between the server 103 and the user computing device 104. The aforementioned elements may interact with one another to create a recipe 109 for a personalized meal in response to the server 103 operating the software program 105, wherein the software program 105 processes the user data set 102 and creates a recipe 109 from recipes 109 stored on memory 112.

The operation of system 100 begins with initial input of a user data set 102 by a user 101, wherein user 101 accesses the system 100 through user computing device 104 that is coupled to server 103. User 101 inputs user data set 102 through input interface on user computing device 104, wherein user computing device 104 transmits user data set 102 to server 103, wherein user data set 102 is stored on memory 112. In some embodiments, user data 102 is collected automatically by server 103 from data recorded in other of user's fitness, nutritional, and other applications maintained on user computing device 104 or other database accessible online. System 100, in some embodiments, may generate nutritional recommendations in a form such as, but not limited to, a recipe 109 with a macronutrient composition 160 based upon biometrics and a goal(s) of user 101. User data set 102 may include the biometrics and goals of user 101, and may represent some of the various independent variables utilized in the algorithms executed by software program 105.

Some examples of algorithms which are useful in calculating personalized values necessary to achieve goal 124, in some embodiments, include formulae for a basal metabolic rate, a resting metabolic rate, a body fat percentage, and a lean muscle mass. Non-limiting examples of biometrics input by user 101 to the user data set 102, in some embodiments, include gender, age, height, weight, activity level, daily caloric and macronutrient intake, and body type. User goal may be a short-term goal or a long-term goal. Some non-limiting examples of a short-term goal include fat loss, such as rapid fat loss, fast fat loss, moderate fat loss, or slow sustainable fat loss; and muscle gain, such as lean muscle gain, medium muscle gain, heavy muscle gain, or maximum muscle gain. Some non-limiting examples of a long-term goal include overall changes in body physique, such as changing from a non-conditioned overweight physique to an athletically conditioned physique with above average muscle mass and/or lower than average total body fat percentage.

In some embodiments, user computing device 104 may be a personal computer, such as a desktop computer, or a laptop computer, or a mobile computing device such as, but not limited to a tablet, a smartphone, a smartwatch, or a similar mobile computing device or wearable. Smartwatches and wearables may constitute and include activity trackers that may be coupled to the user computing device 104 and communicates user activity tracked by the activity tracker to the server 103 to be stored as part of user data set 102.

In embodiments wherein processor 103 is remote from user 101, user data set 102 is uploaded through an input interface, which may include a graphical user interface. For example, in some embodiments, system 100 may include user computing device 104 that is a mobile computing device operating a mobile app or other mobile interface. In embodiments wherein system 100 utilizes a mobile interface as described herein, user 101 may enter food consumed by inputting restaurant food designations and other food data directly into the user's mobile device at the time the food is consumed.

Software program 105, in some embodiments, further comprises coding for macro formulae utilizing the aforementioned formulae; for example, coding to link formulae, formulae for ketogenic macros, formulae for “Paleo Diet” macros, and formulae for “reverse dieting.” In some embodiments, software program 105 automatically generates macros according a user profile which is dependent upon a level of service chosen by user 101; i.e., subscription level.

Software program 105 additionally comprises algorithms for creating custom nutritional programs personalized to an individual user 101, including recipes 109 and meals. Algorithms, in some embodiments, are based upon evidence-based recommendations for nutrition and fitness, such as results of individual prospective-randomized scientific studies or meta-analysis of a group of scientific studies.

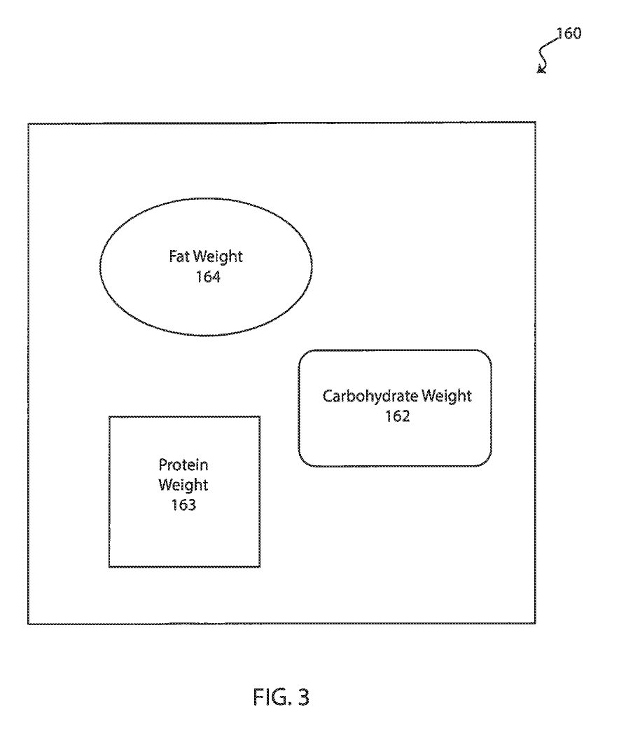

FIG. 3 is a schematic diagramming macronutrient composition 160. A standardized ratio 170 comprises the numerical ratio between carbohydrate weight 162, protein weight 163, and fat weight 164. In some embodiments, standardized ratio 170 is according to an evidence-based formula 130, such as nutritional studies and the like. Software program 105, in response to user data set 102, dictates meal portions to generate a desired macronutrient composition 160. Macronutrient composition 160 comprises a carbohydrate weight 162, a protein weight 163, and a fat weight 164, the absolute values and relative proportions of which are determined by software program 105 in accordance with user data set 102. Server 103 operates software program 105 in to process macronutrient composition 160 utilizing a food selector 108 algorithm, wherein food selector 108 chooses recipe(s) 109 in accordance with macronutrient composition 160.

Food selector 108 selects recipes 109 and meals to provide the desired standardized ratio 170 and macronutrient composition 160 necessary for user 101 to meet his or her goal. Food selector 108, in some embodiments, may access a database of foods, possibly including raw foods, processed foods, and restaurant foods. In some embodiments the user may either provide a list of food he or she likes or dislikes or the user may complete a food selection form, rating each food on a scale from strongly like to strongly dislike. From this database, software program 105 determines options of individual foods and/or combinations of foods and executes food selector 108 to calculate portion sizes based upon a total caloric value 161. In some embodiments, food selector 108 also simultaneously determines recipes 109 necessary to provide total caloric value 161 and macronutrient composition 160 determined by software program 105 based upon user data set 102 for each particular day, and considering a flavor type choice of user 101. Food selector 108 then generates recipe(s) 109 and transmits the same to user computing device 104. In some embodiments, user 101 may select flavor type from which recipes 109 are selected by software program 105. Flavor type, in some embodiments, is part of user data set 102. Some non-limiting examples of flavor types include Mediterranean, Asian, Italian, Tex-Mex, and Southern. In some embodiments, user 101 selects a preferred meal according to a combination of recipes 109 from a flavor type.

In some embodiments, system 100 further comprises a meal preparation service wherein meal(s) are prepared and packaged for later consumption by user 101. In some embodiments, system 100 further comprises a meal delivery service, wherein meal(s) are delivered to user 101.

User computing device 104 may include a visual interface 110, as shown in FIG. 1, which may be a display screen, a touch screen and the like without limitation. Server 103 may automatically generate an avatar 106 and transmit the same to user computing device 104 for display on visual interface 110. Avatar 106 is generated by software program 105 processing independent variables from user data set 102. The appearance of avatar 106 depends upon the particular user data set 102 and transforms with changes in user data set 102 as user 101 progresses through personalized metabolic and nutritional modification system 100. In some embodiments, for example, at proscribed time intervals user 101 takes taped body measurements for input into user data set 102, such as waist circumference, hip circumference upper arm circumference, thigh circumference, and the like. Software program 105 then compiles these and other changes to user data set 102 and transforms the visual appearance of avatar 106 via processor 103 in response to changes in recorded user data set 102. In some embodiments, logic for automatic adjustments is coded into software program 105 along with additional associated programming that links formulae and other calculators to an adjustment logic. Avatar 106 provides visual cues and feedback to user 101 which are useful as positive reinforcement of user's compliance with diet and exercise requirements of system 100, or, conversely, negative reinforcement of noncompliance.

In some embodiments, system 100 further comprises a linking system which uses a user photograph to estimate the body fat composition of user 100, wherein software program 105 links the estimated body fat composition to the visual appearance of avatar 106. User photograph may, for example, be obtained by a user web cam, smartphone camera, or other similarly ubiquitous digital camera device linked to processor 103, either directly or via a data uplink such as a cell phone tower or wired/wireless Internet connection. In some embodiments, user 101 may allow user photograph to be collected automatically from said digital camera device at daily or weekly intervals. In this manner, user photograph becomes part of user data set 102 wherein software program 105 may then utilize user photograph to transform the visual appearance of avatar 106 via processor 103. In some embodiments, user 101 may retrieve a visual image of avatars 106 saved at intervals, such as daily, weekly, or monthly, for example, from a memory available to processor 103. In this example and some other embodiments, user 101 may visualize user 101′s stepwise progression toward his or her goal through a series of saved visual images of transforming avatar 106.

In some embodiments, the color scheme and other aspects of avatar 106 are used for branding, wherein persons, businesses, or other entities may design certain aspects of avatar 106′s appearance, such as facial appearance and clothing, while retaining the general body appearance consistent with that proscribed by system 100 in accordance with user data set 102. In some embodiments, the appearance of avatar 106 may be linked to social networking sites and platforms for sharing with other users, family members, professional colleagues, and the like. Indeed, software program 105 may be adjusted to tie a plurality of psychological, marketing, advertising, and other platforms together with avatar 106 functioning as the visual “hub.”

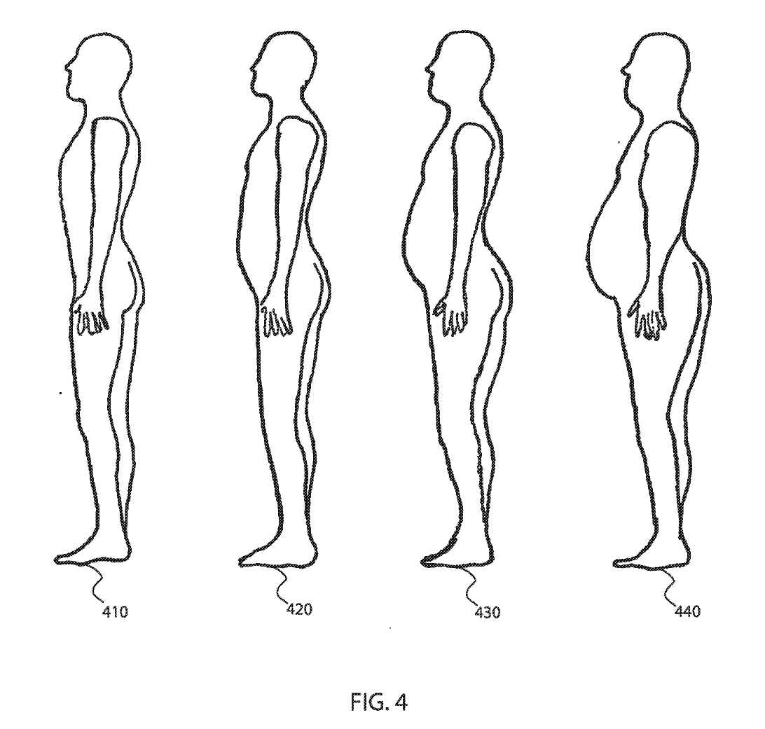

FIG. 4 is a schematic representation of a user avatar for a personalized nutritional system. Profile 410 represents an athletically conditioned avatar 106. An example of user 101 represented by profile 410 include a user of system 100 who has reached a final goal, such as above average muscle mass or leaner than average body fat composition. Profile 440 represents a non-conditioned avatar 106. An example of user 101 represented by profile 440 includes a non-conditioned person with minimal or no exercise experience who may or may not be overweight, or an overweight user 101 with some exercise experience. Profile 430 represents a first transitional avatar 106 and profile 420 represents a second transitional avatar 106. First transitional profile 430 and second transitional profile 420 represents interim transitional states of user fitness as user 101 progresses from entry into program 100 to either completion of program 100 or transition to an embodiment of program 100 which addressed maintenance of a present level of fitness, nutrition, and body composition. The aforementioned profiles provide user 101 with visual feedback and step-wise goals for motivating and challenging user 101 to attain goal 124 through strict compliance with program 100.

It is anticipated that avatar 106 and software program 105 will, generally, develop and evolve in accordance with changes in empirical data and evidence-based interventions associated with health care, including but not limited to exercise, overall physical fitness and nutrition, dietary supplementation, and the like.

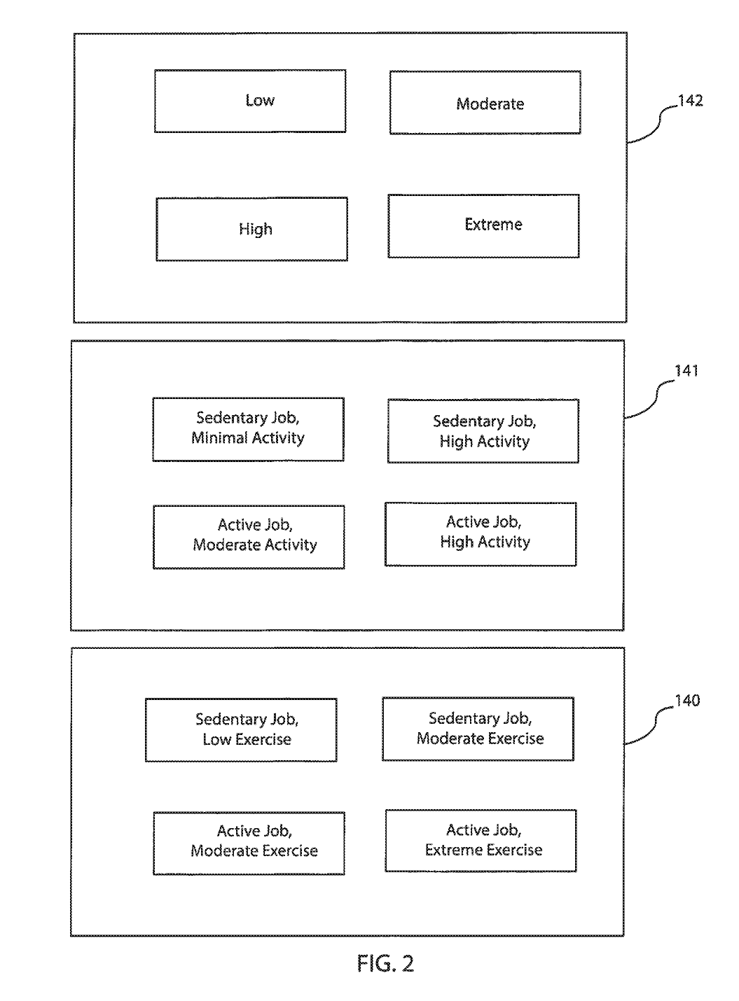

FIG. 2 is a schematic diagramming the relationships between some various possible user activity levels of personalized metabolic and nutritional modification system 100. In some embodiments, user activity level represents an overall user activity 140. As shown in FIG. 2, overall user activity 140, in some embodiments, is determined by combining a work activity 141 and an exercise activity 142. Work activity 141, in some embodiments, is generally characterized as a sedentary job, such as an indoor office-type job; or an active job, such as an outdoor construction job. Exercise activity 142, in some embodiments, is characterized as low activity, moderate activity, high activity, or extreme activity. Some examples of low activity include no exercise or minimal exercise such as housecleaning or light yard work. Some examples of moderate activity include light walking, golf, or light weight training. Some examples of high exercise include fast walking, hiking, running, cycling, weight lifting or circuit training, swimming, or aerobics. Some examples of extreme activity include the aforementioned activities in combination and for long time periods. Accordingly, work activity 141 and exercise activity 142 may be combined to determine overall user activity 140. For example, a user 101 with low exercise activity and a sedentary job would be in one category of overall user activity 140 and a user with an active job with moderate exercise would be in a different category of overall user activity 140. All of the aforementioned lists are by way of example only and in no way meant to be limiting. Following user 101′s entry of user data set 102, a user interface uploads user data set 102 to processor 103.

An embodiment includes a Machine Learning Master Link. The machine learning master link operates to create a link between exercise, nutrition, sleep stress and the like. The machine learning master link comprises a server programmed to receive and automatically store user information, wherein the user information includes meals consumed by the user; timing of meals consumed; exercise performed, including type of exercise, length of exercise, heart rate, and/or calories burned; outcomes for the exercises performed; macronutrient outputs; amount of sleep; interruptions in sleep; stress incurred; and the like.

The server is further programmed to process the user information and create current values associated with the user information. The server is further programmed to automatically compare the values generated regarding the current user information with historical values of user information to determine the effect of the current user information with regard to the historical user information. The server may then be further programmed to create and provide user recommendations in response to the comparison of the current values and the historical values. The user recommendations may include meal recommendations and timing for consuming the recommended meals; recommended exercises, including type of exercise, length of exercise, recommended heart rate, and/or calories burned; macronutrient outputs; recommended amount of sleep; and the like.

By way of example, the machine learning master link provides an effective communication from service to service that creates the overall effect of prescribing the perfect recommendations for the user across every channel. The amount and type of exercise a user is doing has an effect on the intensity recommendations of the exercise which then has an effect on the user's macronutrient output, which then has an effect on the timing and composition of the meals generated by the meal recommender.

The system may further include an exercise recommendation module. The exercise recommendation module operates to establish a system of checks and balances to prevent the system from recommending weights that are too high or low for a given user size. In some embodiments, the exercise recommendation module may include the server programmed to receive and store exercises performed by trained professionals to establish reference points. The exercise recommendation module may include the server programmed to build an automated periodization model for a variety of training protocols. These training protocols may include, without limitation DUP, overreaching, Linear, Step-wise, and the like. In embodiments, the server is programmed to receive and store a user's exercise data, including, without limitation, user exercise data completed, such as repetitions of weight lifting exercises performed and RPE for the prescribed weights and exercises. The exercise recommendation module includes the server programmed to process the data is fed into the system to gauge the user's overall performance and make adjustments and progressions as needed.

The exercise recommendation module may include the server programmed to send to a user computing device an exercise selection interface, wherein the exercise selection interface may include accessory work that can be selected from a menu of exercises that accomplish a similar function. The server may be programmed to create and send to the user computing device, for display, a flow chart or the like to show and/or track progression of the accessory work in order for the server to properly periodize the volume of accessory work to ensure maximal safe progression by the user.

The exercise selection interface may further includes exercise programs that can be selected based on overall fitness goals and objectives of the user, such as, but not limited to “build muscle”, “get stronger”, “athletic performance”, “become more agile”, “get faster”, “run farther”, and the like. The selection of an exercise program may establish the accessory work as described previously.

Embodiments of the machine learning master link and the exercise recommendation module may be utilized for various types of exercise, such as, but not limited to yoga, weight lifting, cardio exercises and the like. With regard to cardio exercises the system may include cardio programs that can track a user's movement and speed during the workout via GPS and communicate the same to the server. The server may be programmed to processes the information and create a notice and send a prompt to the user to speed up or slow down, in response to calculating a measurements for the total distance, average speed, interval speed, and energetic output (this data may be fed back and/or stored in the server to utilize for recommendations). Also, progressions can be measured and adapted to get users closer to a user selected fitness goal.

An additional embodiment includes the use of a pantry and refrigerator database and system for accessing same. The system allows users to use mobile computing devices, such as phones, to scan QR codes on grocery receipts, or any other receipts, or even on a label of a food item that the user has purchased. These items will have the macronutrient composition of each serving, along with total servings, and servings remaining logged into a “pantry” or “refrigerator” database. In an exemplary operation of the system, when a user is at home, the system will know exactly what foods the user has access to as the GPS of the mobile computing device of the user registers the location of the user as “home” and the system accesses the database that corresponds with the food items that are actually in the user's pantry and refrigerator. Each time a user logs that food item, the proper amount of servings is deducted from the total of that container and the same is recorded in the pantry or refrigerator database.

When servings are running low or a food item is almost empty, as tracked in pantry or refrigerator database, the food item will automatically be added to a “grocery list” of the user, particularly if it is a favorite or frequently eaten food item. In some embodiments, the system may communicate with online or other food delivery services to order food items for delivery when particular items are running low as tracked within the database. In some of these embodiments, the system may automatically place an order for that food item and the third party grocery supplier will deliver the required items on the “grocery list”. The system may also be utilized to access recommended recipes and will process data stored in the pantry or refrigerator databases to identify known items the user has access to in the user's home location in the meal generation feature. Further, in some embodiments, the system 100 may include the machine learning master link programmed to copy a tracked food item or meal, and then insert or otherwise paste the item to any number of day(s) into the future for establishing a future meal plan. This may be done automatically wherein a selected item may be automatically inserted within specific selected days of a meal calendar.

The system may include an “on the go” food/meal recommendation component. In operation of the “on the go” component, if a user is away from home, a GPS feature can be used to locate restaurants that are nearby and automatically scans the restaurants' menu information to recommend a restaurant for the user to eat at and determines particular meals to order that will fit the user's macronutrient targets. This “on the go” component may also communicate with the Avatar macronutrient tracking and meal generation part of the system. The system may also utilize the GPS feature to determine navigation to and give directions to a selected restaurant by the user. The system may also be established to automatically direct a user to the nearest place that fits the user's preferences and suggests the meal to order.

The system may include an Avatar Exercise component for linking with yoga and every other form of exercise. The Avatar Exercise application or component will link with all other components of the system, wherein on more intense training days, the macronutrient recommendations are automatically adjusted in response to the more intense training days. Additionally, the Avatar Exercise component may link with other supplementary applications or components like Avatar Yoga, wherein the stretching routines and recovery routines suggested will be targeted to the proper muscle groups that were worked in response to the exercises performed using the Avatar Exercise application or component.

The machine learning component of the system operates with all of the different components of the system as they relate and/or correlate to one another in order to make the best recommendations and programs possible specific to each user.

This invention overcomes the aforementioned and other difficulties encountered with using prior art, like the need of a personalized, evidence-based, automated metabolic and nutritional modification system, which provides feedback in the form of reinforcement to the user, for example. The personalized nutritional and metabolic modification system is designed to create a practical, efficient, evidence-based means of providing the user the proper information, food, supplements, and exercise plan to meet the user's health and fitness goals. The system changes in response to the user's progression toward a defined goal, with a transforming avatar visualized by the user. The widespread availability of a personalized nutritional and metabolic modification system will aid persons seeking to lose weight, improve function and overall athletic ability, serious athletes, persons with nutritionally related chronic diseases, and other health-related problems to optimize their overall health and level of function. This invention also has applications for use in many other situations requiring a safe, scientifically based approach to improving overall health and fitness.

Exceptional results can be obtained with the personalized nutritional and metabolic modification system described in the various embodiments of the invention. The system is intuitive, safe, and easy to use. Thus, the various disclosed embodiments of the personalized nutritional and metabolic modification system have immediate applicability in the healthcare and personal fitness industries, and similarly across other applications.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A personalized nutritional and metabolic modification system comprising:
a user computing device for accessing the system, the user computing device comprising a visual interface, wherein the visual interface displays a graphical user interface for user interaction with the system including data input; and
a computer server at a service provider, the computer server containing a machine learning master link, which computer server is coupled to the user computing device and the machine learning master link is programmed to:
receive and automatically store current user information, wherein the current user information includes one of meals consumed by the user; timing of meals consumed; exercise performed, including type of exercise, length of exercise, heart rate, and/or calories burned; outcomes for the exercises performed; macronutrient outputs; amount of sleep; interruptions in sleep; stress incurred; or combinations thereof;
process the user information and create current values associated with the current user information;
automatically compare the current values generated regarding the current user information with historical values created from historical user information to determine the effect of the current user information with regard to the historical user information; and
create and provide a user recommendation in response to the comparison of the current values and the historical values.

2. The system of claim 1, wherein the user recommendation includes a meal recommendation and timing for consuming the recommended meals.

3. The system of claim 2, wherein the machine learning master link is further programmed to copy a tracked meal of the recommended meals and automatically insert the copied tracked meal into future day meal calendar.

4. The system of claim 1, wherein the user recommendation includes recommended exercises.

5. The system of claim 4, wherein the recommended exercises includes a type of exercise, a length of exercise, a recommended heart rate, and/or calories to burn.

6. The system of claim 1, wherein the user recommendation includes macronutrient outputs.

7. The system of claim 1, wherein the user recommendation includes recommended amount of sleep.

8. The system of claim 1, wherein the user recommendation includes more than one user recommendation.

9. The system of claim 8, wherein the more than one user recommendation includes at least two recommendation including meal recommendations and timing for consuming the recommended meals; recommended exercises, including type of exercise, length of exercise, recommended heart rate, and/or calories to burn; macronutrient outputs; and recommended amount of sleep.

10. A personalized nutritional and metabolic modification system comprising:
a user computing device for accessing the system, the user computing device comprising a visual interface, wherein the visual interface displays a graphical user interface for user interaction with the system including data input; and
a computer server at a service provider, the computer server containing exercise recommendation module, which computer server is coupled to the user computing device and the exercise recommendation module is programmed to:
receive and store exercises performed by trained professionals to establish reference points;
build an automated periodization model for a variety of training protocols;
receive and store a user's exercise data;
process the data is fed into the system to gauge the user's overall performance and make adjustments and progressions; and
send to the user computing device an exercise selection interface, wherein the exercise selection interface may include exercise programs and accessory work that can be selected from a menu, wherein the exercise programs are based on overall fitness goals and objectives of the user comprising at least one of “build muscle”, “get stronger”, “athletic performance”, “become more agile”, “get faster”, “run farther”, and combinations thereof.

11. The system of claim 10, wherein the exercise recommendation module is further programmed to create and send to the user computing device, for display, a tracking interface to show and track progression of the accessory work in order for the server to properly periodize the volume of accessory work to ensure maximal safe progression by the user.

12. The system of claim 10, wherein the exercise recommendation module is programmed to automatically establish the accessory work in response to selection of the exercise program.

13. The system of claim 10, wherein the training protocols include one of DUP, overreaching, Linear, Step-wise, and combinations thereof.

14. The system of claim 10, wherein the user exercise data includes one of repetitions of weight lifting exercises performed, RPE for the prescribed weights and exercises, yoga exercise performed, distances run, or combinations thereof.

15. A personalized nutritional and metabolic modification system comprising:
a user computing device for accessing the system, the user computing device comprising a visual interface, wherein the visual interface displays a graphical user interface for user interaction with the system including data input; and
a computer server at a service provider, the computer server containing a machine learning master link and an exercise recommendation module, which computer server is coupled to the user computing device and wherein:
the machine learning master link is programmed to:
receive and automatically store current user information, wherein the current user information includes one of meals consumed by the user; timing of meals consumed; exercise performed, including type of exercise, length of exercise, heart rate, and/or calories burned; outcomes for the exercises performed; macronutrient outputs; amount of sleep; interruptions in sleep; stress incurred; or combinations thereof;
process the user information and create current values associated with the current user information;
automatically compare the current values generated regarding the current user information with historical values created from historical user information to determine the effect of the current user information with regard to the historical user information; and
create and provide user recommendations in response to the comparison of the current values and the historical values; and
the exercise recommendation module is programmed to:
receive and store exercises performed by trained professionals to establish reference points;
build an automated periodization model for a variety of training protocols;
receive and store a user's exercise data;
process the data is fed into the system to gauge the user's overall performance and make adjustments and progressions; and
send to the user computing device an exercise selection interface, wherein the exercise selection interface may include exercise programs and accessory work that can be selected from a menu, wherein the exercise programs are based on overall fitness goals and objectives of the user comprising at least one of “build muscle”, “get stronger”, “athletic performance”, “become more agile”, “get faster”, “run farther”, and combinations thereof.

16. The system of claim 15, wherein the user recommendations include one of meal recommendations and timing for consuming the recommended meals; recommended exercises, including type of exercise, length of exercise, recommended heart rate, and/or calories burned; macronutrient outputs; recommended amount of sleep; or combinations of two or more thereof.

17. The system of claim 15, wherein the exercise recommendation module is further programmed to create and send to the user computing device, for display, a tracking interface to show and track progression of the accessory work in order for the server to properly periodize the volume of accessory work to ensure maximal safe progression by the user.

18. The system of claim 15, wherein the exercise recommendation module is programmed to automatically establish the accessory work in response to selection of the exercise program.

19. The system of claim 15, wherein the training protocols include one of DUP, overreaching, Linear, Step-wise, or combinations thereof

20. The system of claim 15, wherein the user exercise data includes one of repetitions of weight lifting exercises performed, RPE for the prescribed weights and exercises, yoga exercise performed, distances run, or combinations thereof.
